(19) United States
(12) Patent Application Publication
(10) Pub. No.: US 2017/0303980 A1
(43) Pub. Date: Oct. 26, 2017

(54) Natural Polymer-Based Porous Orthopedic Fixation Screw for Bone Repair and Regeneration

(71) Applicant: University of Connecticut, Farmington, CT (US)

(72) Inventors: Sangamesh G. Kumbar, Vernon, CT (US); Cato T. Laurencin, Avon, CT (US)

(21) Appl. No.: 15/649,044

(22) Filed: Jul. 13, 2017

Related U.S. Application Data
(63) Continuation of application No. 14/859,908, filed on Sep. 21, 2015, now abandoned.
Application No. 14/859,908 is a continuation of application No. 13/033,094, filed on Feb. 23, 2011, now abandoned.
(60) Provisional application No. 61/307,137, filed on Feb. 23, 2010.

(51) Int. Cl. A61B 17/86; A61L 31/04; A61L 31/12; B29C 39/02; B29C 39/38; B29C 67/04; B29C 67/24; B29L 31/00; A61B 17/00; B29K 1/00; B29K 105/00

(52) U.S. Cl. CPC A61B 17/866; B29C 67/04; A61L 31/042; A61L 31/125; B29C 39/02; B29C 39/38; B29C 67/24; A61B 2017/00004; A61B 2017/00526; B29K 2001/08; B29K 2001/12; B29K 2005/00; B29K 2105/251; B29L 2031/7546

(57) ABSTRACT

A bone fixation device made of polysaccharide particles or microspheres fused into a solid structure is provided herein. The bone fixation device may be in the form of an orthopedic screw, orthopedic pin, or orthopedic plate. Methods of making the bone fixation devices described herein are provided as are methods of treating patients in need of bone repair or replacement by implanting a bone fixation device described herein in the patient at a site of bone damage, ligament damage, or bone deformity.

### CROSS REFERENCE TO RELATED APPLICATION

This application claims priority from U.S. Provisional Patent Application No. 61/307,137, filed Feb. 23, 2010, which is hereby incorporated by reference in its entirety.

### FIELD OF THE INVENTION

A bone fixation device made of polysaccharides is provided. In certain embodiments the bone fixation device is an orthopedic screw, orthopedic pin, or orthopedic plate. One embodiment provides natural polymer-derived interference screws for use in graft fixation in anterior cruciate ligament (ACL) reconstruction. Methods of making the bone fixation devices described herein are provided. Also provided are methods of treating patients in need of bone repair or replacement by implanting a bone fixation device described herein in the patient at a site of bone damage, ligament damage, or bone deformity.

### BACKGROUND

The repair and replacement of damaged hard tissues such as bone is a major clinical problem in the U.S. and around the world. In the U.S. alone, more than 500,000 hip and knee replacements are performed and over a million fractures are treated each year (Bucholz, Clin. Orthop. (2002) 398: 44-52). These numbers are expected to grow as the US population grows and the life expectancy of the population increases. Current bone replacement procedures often use autograft or allograft tissue but these approaches have limitations. Autograft tissue is often limited in supply and carries the potential for donor site morbidity. Allograft tissue carries the potential for disease transmission and immunological rejection (Mankin, Clin. Orthop. Relat. Res. (2005) 432: 210-216).

The field of tissue engineering seeks to design tissue substitutes for clinical use to replace diseased organs or to heal and regenerate damaged tissue. The tissue engineering approach holds potential for overcoming the limitations associated with the use of autografts and allografts. Scaffold based tissue engineering has become a promising strategy to regenerate three-dimensional (3-D) tissues for transplantation. In the scaffold approach a three dimensional framework, or scaffold, is constructed and inserted at the tissue damage site. The scaffold then provides a surface for the attachment and re-growth of biological tissue. A three-dimensional bioresorbable porous construct with appropriate mechanical properties is required to guide cellular attachment and subsequent tissue formation (Borden, et al., Biomaterials, (2002) 23: 551-559; Katti and Laurencin, in Advanced Polymeric Biomaterials, Shonaike and Advani (eds.) CRC Press, Boca Raton, 2005, 484-527; Uhrich, et al., Macromolecules, (1995) 28: 2184-2193; Kumbar, et al., J. Inorg. Organometallic Polym. Mater. (2006) 16: 365-385; and Kofron, et al., J. Biomed. Mater. Res. A. (2007) 82: 415-425).

A wide range of synthetic and natural polymers have already been adopted for 3-D scaffold fabrication. Synthetic biodegradable polymers such as poly(esters), poly(anhydrides), poly(anhydride-co-imides) and poly(phosphazene) derivatives, have been used to fabricate scaffolds for bone repair. These synthetic materials been investigated as potential candidates for scaffold fabrication due to their programmable degradation characteristics (Laurencin, et al., in Annual Review of Biomedical Engineering, Yarmush (ed.) Annual Reviews Inc., Palo Alto, (1999) 1: 19-46). The α-hydroxyesters poly(lactic acid) (PLA), poly(glycolic acid) (PGA) and their copolymer PLAGA are approved by Food and Drug Administration (FDA) for certain biomedical applications (Athanasiou, et al., Arthroscopy, (1998) 14: 726-737).

The utility of synthetic scaffold materials in transient biomedical applications, including implants, is hampered due to the acidic degradation products that can adversely affect the biocompatibility (Taylor, et al., J. Appl. Biomaterials, (1994) 5: 151-157). This problem becomes more acute in larger sized implants or at implant sites with minimal fluid flow. For instance, in articular cartilage the acidic degradation products can accumulate significantly and affect the cells and the tissues surrounding the implant. A clinical study involving 1000 patients over a period of 9 years reported that a significant number of patients developed inflammatory foreign body reaction to implants that were made of PGA, PLA and PLAGA. Some of the patients developed severe osteoarthritis in the joints near the implants and some of these had to undergo arthromeres (Bostman, et al., J. Bone Joint Surg., (1998) 80: 333-338). The drawbacks of hydroxyesters underscore the need for new polymers for biomedical applications that degrade into non-toxic, non-acidic, non-immunogenic byproducts, while provided the required mechanical stability.

Scaffolds derived from the polymers of natural origin have shown superior biological performance due to their chemical similarity with the extracellular matrix (ECM) components, which the biological environment is prepared to recognize and deal with metabolically. Natural polymer scaffolds may also avoid the stimulation of chronic inflammation or immunological reactions and toxicity often associated with synthetic polymer scaffolds. However, the currently available scaffolds fabricated from natural origin material do not possess adequate mechanical properties, interconnected pore structure and/or porosity for bone healing applications at load bearing sites.

During graft fixation surgery, such as ACL reconstructive surgery, interference screws are used to secure a bone graft to a bore in a bone mass. Currently, metal and polymer based interference screws are used for orthopedic applications. Metal screws are permanent and known to cause stress concentration that sometimes weakens the bone to which the graft is affixed. Additionally repetitive microstrains at the bone-implant interface during function can lead to implant failure. These concerns, and the inconvenience of screw removal during revision and other subsequent surgeries in the knee, led to the development of bioabsorbable interference screws for graft fixation. Current bioabsorbable interference screws are based on α-hydroxyesters; poly(lactic acid) (PLA), poly(glycolic acid) (PGA) and their copolymer PLAGA.

Mechanical tests and clinical outcomes have shown that the bioabsorbable interference screws are equivalent to metal screws in ACL graft fixation. However, there are reports concerning the in vivo degradability and biocompatibility, as well as the osseous replacement of the implants. For example, several experimental studies have been performed to investigate tissue response and tissue replacement after implantation of PLLA (poly-L-lactic acid) material into bone. Unfortunately, their follow-up of 48 to 52 weeks was inappropriate to evaluate either tissue response or tissue replacement, because little or no signs of material degradation had taken place. MRI scans of patients who had been implanted with high molecular weight PLLA screws to stabilize ankle fractures showed no osseous replacement of the implant had occurred up to 6 years after implantation. The process of osseous replacement may require several years even for faster-degrading implants if there has been evidence of an osteolytic lesion during the final stage of degradation. Clinical reports have shown that remnants of high molecular-weight PLLA implants could still be found several years after implantation. These reports suggest that a complete degradation of highly crystalline, so-called biodegradable, implants does not occur within an appropriate time.

The usefulness of current bioabsorbable interference screws is also hampered due to acidic degradation products that can adversely affect the biocompatibility. Hence there is a need to develop alternate bioabsorbable interference screws that degrade into non-toxic and non-immunogenic byproducts. The bioabsorbable orthopedic fixation screws, including interference screws, provided herein fulfill this need and provide additional advantages described herein.

### SUMMARY

A bone fixation device made of polysaccharides is provided. The polysaccharide used in the bone fixation device may be in the form of microspheres or particles comprising derivatized celluloses, for example comprise ethyl cellulose and/or cellulose acetate, and the polysaccharide microspheres may have a microsphere diameter of about 100 micrometers to about 1200, or of about 300 micrometers to about 600 micrometers and the polysaccharide particles may have a diameter of about 50 to about 500 micrometers, of about 50 to about 400 micrometers, or about 50 to about 150 micrometers. The bone fixation device optionally includes one or more of collagen nanofibers, hydroxyapatite, or β TriCalcium Phosphate (TCP).

In certain embodiments the device is an orthopedic screw, an orthopedic pin, or an orthopedic plate. In an embodiment the orthopedic screw, such as an interference screw, comprises a threaded portion having a proximal and distal end and a tip disposed on the distal end. In an embodiment the orthopedic plate provided herein, is structured to be secured to the bone so that the plate covers an exterior surface region of the bone, and the plate may additionally have openings through which the plate is secured to the bone.

A method of making a bone fixation device is included herein comprising providing a plurality of polysaccharide particles or microspheres in a mold, wherein the mold is in the form of the bone fixation device; providing a solvent system having an organic solvent fraction and an aqueous fraction dropwise in the mold; removing excess solvent from the mold; and fusing the particles into a solid structure in the mold. The bone fixation device may also be made by injection molding.

A method of treating a patient in need of bone repair, including ACL graft fixation is included in this disclosure. Interference screws used to secure the ACL graft in the femur and tibia are also provided by this disclosure. The method of treatment optionally includes first excising damaged or deformed bone from the patient.

### BRIEF DESCRIPTION OF THE DRAWINGS

FIG. 1. Design and fabrication of microparticle-based interference screws made from cellulose and its derivatives such as cellulose acetate (CA 30K and 50K molecular weight) and ethyl cellulose (EC). Screw is shown with dimensions of 6.2 mm (OD) and 18.2 mm (length).

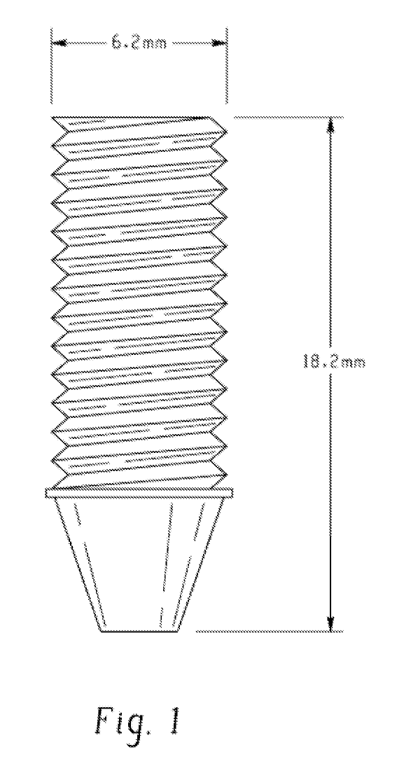

FIG. 2. Representative stress-strain curves of the CA screws (5×10 mm) fabricated from the particles in the diameter range of 50-400 μm of (A) 30,000 and (B) 50,000 Mn under compression. The compressive modulus values were found to be 227±59 and 292±40 MPa for the scaffolds fabricated with 30K and 50K respectively. Observed stress-strain behavior is similar to that of native bone identified by a linear elastic region and less stiff post-yield region. Increase in molecular weight further improves the mechanical properties due to restricted polymer chain moment with higher molecular weight. Polysaccharide scaffolds showed compressive mechanical properties in the mid range of human trabecular bone (20-900 MPa) and ideally suited for bone tissue engineering applications.

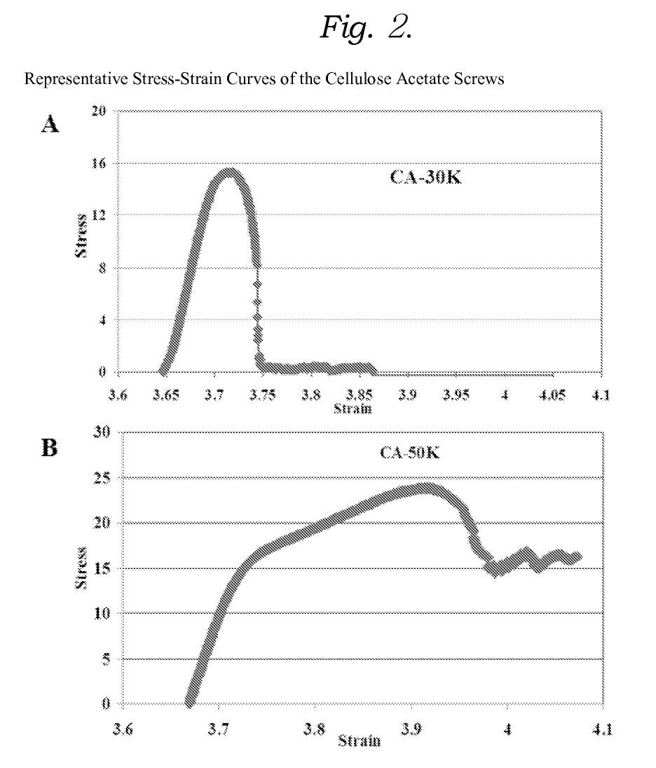

FIG. 3. Quantitative analysis of the CA screw mechanical properties fabricated from the particles in the diameter range of 50-400 μm of 30,000 and 50,000 Mn under compression where (A) Maximum compressive load, (B) Compressive modulus, (C) Compressive strength, and (D) Toughness. It was demonstrated that higher molecular weight screws were tougher with better mechanical properties.

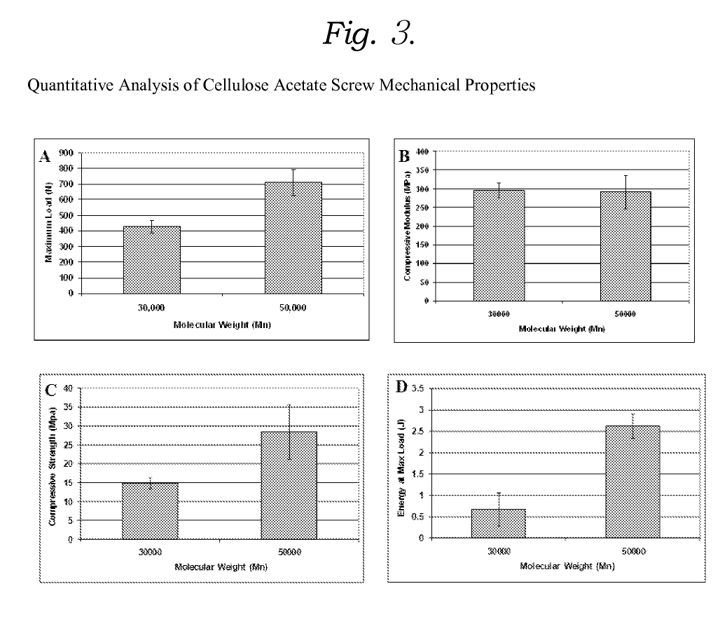

FIG. 4. Representative stress-strain curves of the 15 wt % HA (hydroxyapatite) loaded CA screws (5×10 mm) fabricated from the particles in the diameter range of 50-400 μm of (A) 30,000 and (B) 50,000 Mn under compression. The compressive modulus values were found to be 389±16 and 395±20 MPa for the scaffolds fabricated with 30K and 50K respectively. Observed stress-strain behavior is similar to that of native bone identified by a linear elastic region and less stiff post-yield region. Increase in molecular weight further improves the mechanical properties due to restricted polymer chain moment with higher molecular weight. Polysaccharide scaffolds showed compressive mechanical properties in the mid range of human trabecular bone (20-900 MPa) and ideally suited for bone tissue engineering applications.

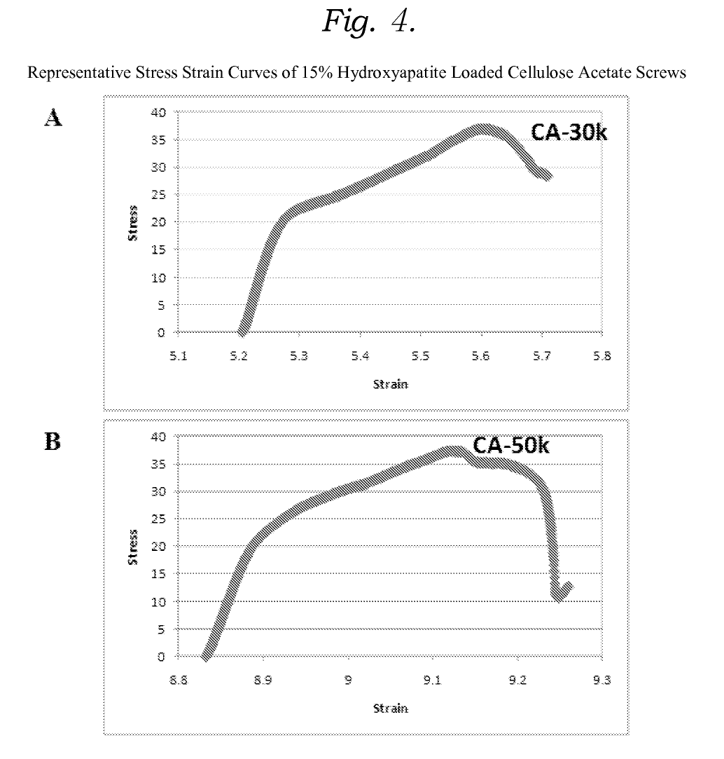

FIG. 5. Quantitative analysis of the mechanical properties of HA-CA (50,000 Mn) composite screws fabricated with varying weight compositions of 10 and 15% HA under compression: (A) Maximum compressive load, (B) Compressive modulus, (C) Compressive strength, and (D) Toughness. Among the various CA to HA compositions (2.5-40 wt %), 15% HA loaded particles and their screws resulted in higher compressive mechanical properties due to a homogenous distribution of nano-sized HA particles. At other compositions mechanical properties were weaker. All these values are in the mid range of human trabecular bone.

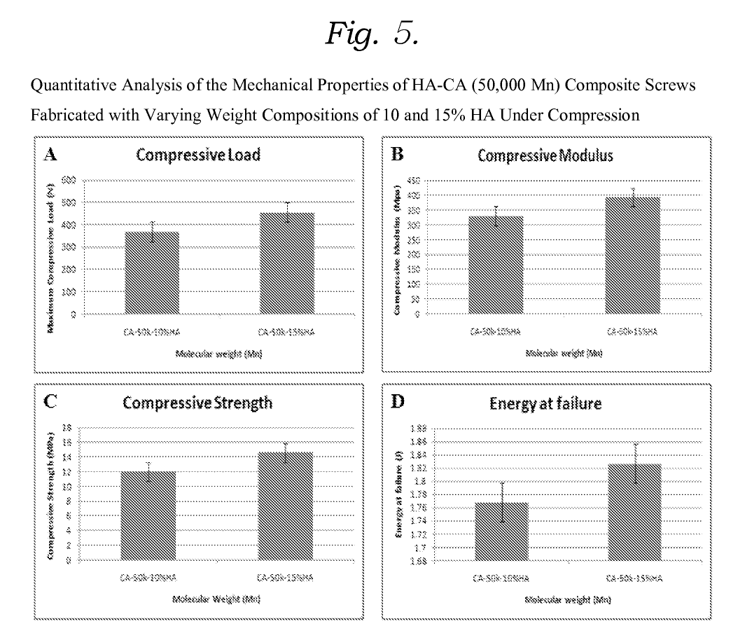

### DETAILED DESCRIPTION

### Terminology

The following terminology may be helpful before considering the detailed description of the invention, which follows.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

In all occurrences where the word “about” appears with a range, e.g. “about 300 micrometers to about 600 micrometers” the exact range is also included, in which the word about does not appear. Thus the invention also pertains to microspheres of 300 micrometers to 1200 micrometers.

The word “comprising” appears the language is meant to be open-end as is it commonly understood to be in patent claims; the inclusion of additional elements is contemplated. In all occurrences where the word “comprising” is used the invention also includes embodiments in which the less open language “consisting essentially of” or “consisting of” can be used.

“Derivatized cellulose” is cellulose that has been chemically modified, either naturally or synthetically. Derivatized cellulose, as used herein, is a polysaccharide derivative. Derivatized cellulose includes, but is limited to methyl cellulose, ethyl cellulose, carboxy methylcellulose, hydroxyethyl cellulose, hydroxypropyl methylcellulose, ethyl methylcellulose, etc. and cellulose acetate.

“Particles” may be of any shape, while “microspheres” are spherical.

“Polysaccharides” are polymers comprised of many monosaccharides joined together by glycosidic bonds. As used herein “polysaccharides” include both natural polysaccharides, such as cellulose and chitin, and synthetic polysaccharide derivatives, such as derivatized cellulose.

An “Interference screw” is a screw for anchoring a flexible transplant such as a tendon or ligament in an opening in a bone.

“Sintering” is the thermal treatment of a powder or compact at a temperature below the melting point of the main constituent, for the purpose of increasing its strength by bonding together of the particles. “Sintered” materials are any materials that have been formed by the process of sintering.

A “Solvent/non-solvent composition” is a solvent system having at least two fractions—a volatile organic fraction (the solvent) and a non-volatile, typically aqueous, fraction. A preferred embodiment is a solvent/non-solvent composition having an organic solvent fraction and an aqueous (non-solvent) fraction. Appropriate solvent fractions include, but are not limited to, acetonitrile, acetone, hexanes, dichloromethylene, methanol, ethanol, and methylethylketone. Solvent/non-solvent compositions include acetone:water (e.g. 3:1) and acetonitrile:water (e.g. 8:1).

### Bone Fixation Devices

Bone fixation devices provided herein; include orthopedic screws, pins, and plates. The bone fixation devices can be formed from sintered microspheres in which the microspheres are polysaccharide microspheres. The bone fixation devices proved herein may also be prepared from polysaccharides particles melted together in a solid mold or by injection molding. Polysaccharide microspheres and particles, comprised of cellulose acetate and ethyl cellulose of varying molecular weights are suitable materials from which the bone fixation devices may be prepared.

The orthopedic fixation devices designed on the polysaccharide platform have several advantages over other polymer-based orthopedic fixation devices: 1) superior mechanical properties that allow the polysaccharide platform to be used for any orthopedic load-bearing application; 2) fabrication is relatively simple, fast, scalable, and carried out in room temperature; 3) ability to incorporate growth factors/antibiotics in the device design during fabrication and their subsequent release; 4) highly biocompatible, currently cellulose and its derivatives are used for a variety of biomedical applications; 5) degradation products are easily metabolized due to structural similarities with native extracellular matrix (ECM) components; 6) degradation process is somewhat slower than for other polymer-based orthopedic fixation devices allowing for more complete osteogenesis, however degradation times are also adjustable by chemical modification, and, 7) cellulose and derivatives are inexpensive and commercially available in all the grades.

Naturally occurring polysaccharides such as chitosan, alginates, cellulose and starch have been extensively used for variety of biomedical applications including scaffolds for tissue engineering applications. Natural polymers offer the advantage of being similar to biological macromolecules, which the biological environment can readily recognize and degrade through metabolic processes. Thus natural polymers may also avoid the stimulation of chronic inflammation or immunological reactions and toxicity, often detected with synthetic polymers. Polysaccharides are derived from renewable resources such as plants, animals and micro-organisms, and are therefore widely distributed in nature. Cellulose, the primary structural component of plant cell walls, is a linear polysaccharide of D-glucose units linked by β(1-4) glycosidic bonds. The fully equatorial conformation of β-linked glucose residues stabilizes the chain structure, minimizing its flexibility. This highly cohesive, hydrogen-bonded structure gives cellulose fibers exceptional strength and makes them water insoluble despite their hydrophilic nature. Fiber scaffolds derived from cellulose have been found useful for cardiac and cartilage tissue engineering. Cellulose sponges have also been reported to be biocompatible and biodegradable for bone tissue engineering applications.

High strength materials such as cellulose are ideal orthopedic fixation devices, including interference screws for ACL reconstruction surgeries, to secure the bone graft to the bone mass. For example in ACL surgery interference screws are used to secure the graft between the femur and tibia. Cellulose based orthopedic fixation screws represent a new generation of bioactive and biofunctional orthopedic fixation screws for bone graft surgery, including interference screws for ACL reconstruction. Cellulose based orthopedic fixation screws promote and foster the growth of surrounding bone tissue, as well as limit any potential problems a patient may incur due to having these screws in his or her body. Use of certain polysaccharides such as cellulose also permit control of orthopedic fixation screw parameters including screw size and screw geometry (amount of tapering, size and angle of threads and the shape of the tip).

The bone fixation devices provided herein comprise polysaccharide particles or microspheres melted into a solid structure. In certain embodiments the polysaccharide microspheres/particles are cellulose derivatives, for example the polysaccharide microspheres may be cellulose or ethyl cellulose. The orthopedic fixation devices can be prepared using the solvent/non-solvent sintering method described in example 1, below, for preparing polysaccharide bone repair scaffold, by melting polysaccharide particles or microspheres, for example at a temperature of about 180° C. to about 240° C., or in some embodiments at about 190° C. Those of skill in the art will recognize certain changes, which are a matter of routine optimization, may be needed.

The orthopedic fixation devices can also be prepared by solvent-assisted melting. Cellulose acetate particles, e.g., in the diameter range of 100-150 μm, are produced from solutions of CA using a water-in oil emulsion/solvent evaporation. Teflon molds were tightly filled with CA particles and a solvent composition of 3:1 ratio of Acetone:Cyclohexane was added to the mold to melt the particles to produce fixation devices at the room temperature. Bone fixation devices are taken out of the mold after 3 hours and kept desiccated until further use. This approach is attractive for delivering growth factors and antibiotics to achieve better graft fixation due to room temperature fabrication.

In an alternate embodiment, bone fixation devices are fabricated from sintered polysaccharide microspheres, including EC or CA microspheres, using an appropriately shaped Teflon mold. In brief, CA particles in the size range of 300-500 μm are tightly filled in the mold and THF solvent is added drop wise to soak the microspheres. Similarly EC microspheres are sintered using cyclohexane:acetone (2:1) ratio. Excess of solvents are drained from the mold by keeping the mold inclined in a fume hood. Bone fixation devices are further dried by applying vacuum in a dessicator for an additional 20 min. Bone fixation devices may also be prepared by melting together polysaccharide particles, including EC or CA particles, in a solid mold, or by injection molding. Particles are typically in the size range of 50-500 μm, or preferably 50-150 μm.

Also included herein are bone fixation devices comprising polysaccharide composites with β TriCalcium Phosphate (TCP) and hydroxyl apatite (HA). Specifically included herein are CA or EC bone fixation devices comprising about 15% TCP/HA. More specifically the TCP:HA ratios of 1:1, 3:1 and 1:3. Combining polysaccharides with osteoconductive materials such as TCP and HA can improve the mechanical properties of the sintered polysaccharide as well as in vivo osteointegration. In brief, CA and EC are mixed with 10-40% (wt/wt) varying amounts of TCP/HA to produce composite particles. These particles are sintered or melted together in a mold as explained in the previous step. These composite material bone fixation devices are designed for their morphology and mechanical properties as explained earlier. The mechanical properties and degradation pattern of the bone fixation devices provided in this disclosure can easily be varied by altering the material composition. Polysaccharide-TCP/HA-composite particles are used to fabricate orthopedic fixation screws, including interference screws, and orthopedic pins and plates. These devices are characterized for their morphology and mechanical performance in various modes as explained below. Additionally, composite scaffolds are characterized for their ceramic (TCP/HA) content using thermo gravimetric analysis (n=6).

Also included herein are polysaccharide bone fixation devices in which the polysaccharide backbone has been chemically modified. Polysaccharide bone fixation devices in which the polysaccharide has been oxidized with periodate or treated with 2,2,6,6-tetramethyl-piperidin-1-yl)oxyl (TEMPO) are included in this disclosure. Such treatment alters the degradation properties of the polysaccharide bone fixation devices.

Further included herein are polysaccharide-based bone fixation devices seeded with a mesenchymal stem cells. The cells may be in the form of a mineralized matrix. The purpose of seeding bone fixation devices with mesenchymal stem cells is to stimulate osteogenesis at the implantation site.

Bone fixation device morphology is characterized by SEM. Bone fixation devices (n=3) are coated with gold using a Hummer V sputtering system (Technics, Baltimore, Md.) for 5 min. Samples are visualized on a JSM 6400 (JOEL, Boston, Mass.) at 15-20 keV at a working distance of 39-48 cm.

Bone fixation device mechanical properties are assessed via the following methods. In one analysis the mechanical properties of an orthopedic fixation screw (n=6) at a length to diameter ratio of 18×6 mm is used for mechanical testing in compression. These screws are also tested in various modes of bending and loading cycles of different compression. An Instron Testing Apparatus (model 5544; Instron, Canton, Mass.) is used at a ramp speed of 1 mm/min at ambient temperature, humidity and pressure until implant failure. Load and displacement will be recorded to plot a stress versus strain curve. For each specimen, (1) compressive modulus (the slope of the linear region of the stress versus strain curve), (2) compressive strength (the magnitude of the maximum force applied divided by the original cross-sectional area), (3) maximum compressive load (the maximum force applied) and (4) the energy absorbed at failure (the area under the stress-strain curve at the point of failure) are be calculated. These results are compared with similar commercially available polyester based devices.

U.S. Provisional patent application 61/154,582 filed Feb. 23, 2009 and U.S. patent application Ser. No. 12/710,637, filed Feb. 23, 2010 are hereby incorporated by reference for its teachings regarding bone repair and replacement materials. The materials described in the '582 application may be used in the present orthopedic fixation devices.

### Orthopedic Screws

Orthopedic fixation screws designed on the polysaccharide platform have several commercial advantages: 1) superior mechanical properties compared to polyester screws; 2) fabrication is relatively simple, fast, scalable, and carried out in room temperature; 3) ability to incorporate growth factors/antibiotics in the screw design during fabrication and their subsequent release; 4) highly biocompatible, currently cellulose and its derivatives are used for a variety of biomedical applications; 5) degradation products are easily metabolized due to structural similarities with native extracellular matrix (ECM) components; and, 6) cellulose and derivatives are inexpensive and commercially available in all the grades.

In one embodiment an orthopedic fixation screw is cannulated and has a tapered profile. Tapered profile makes the screw easy to insert while providing superior fixation resulting from a progressively increasing diameter. In one embodiment the fixation screw includes an Allen wrench style hole in the screw head for driving mechanism ensuring no slippage. Scaffold morphology is characterized using microscopy. Mechanical properties such as compressive modulus and strength, maximum compressive load, and the energy absorbed at failure are evaluated using an Instron Testing Apparatus.

Included herein are orthopedic fixation screws comprising polysaccharide having an OD of about 6.2 mm and a length of about 18.2 mm and orthopedic fixation screws having and OD of 3 mm and a length of about 9 mm.

In one embodiment the orthopedic screw comprises a threaded portion having a proximal and distal end and a tip disposed on the distal end.

### Assessment of Apatite-Forming Ability

Polysaccharide and composite bone fixation devices are assessed for their ability to form apatite layer by incubating them in simulated body fluid. Extent of apatite layer formation is a preliminary confirmation the bone fixation device's ability to integrate with native bone. To test the bioactivity of the bone fixation devices, both the composite and pure polysaccharide bone fixation devices are incubated in simulated body fluid for 28 days. Control devices are incubated in distilled de-ionized (DDI) water. The solutions are changed every other day. After incubation of 7, 14, 21, and 28 days, samples (n=3) are removed from solutions, washed with DDI water, and air-dried for further analysis. The surface morphology and calcium deposition are examined by SEM and alizarin red staining respectively to estimate extent of apatite formation.

### Device Degradation

Polysaccharide and composite bone fixation devices are subjected to degradation in a 37° C. water bath to simulate in vivo conditions. One set of devices is also subjected to cellulase enzyme catalyzed degradation under similar conditions. Changes in molecular weight and net scaffold weight loss over the different time points are measured. To measure changes in molecular weight, cylindrical microsphere scaffolds (n=3) are dissolved in 1% tetrahydrofuran (THF) (w/v). The solution is filtered through a 0.45μ polypropylene filter and analyzed using gel permeation chromatography (model 1100; Hewlett Packard) equipped with a Zorbax PSM 300S column preheated to 40° C. THF is used as the mobile phase at a flow rate of 1 mL/min. Polystyrene standards (Polymer Laboratories, Amherst, Mass.) is used for calibration.

Bioactive Agent Delivery from Bone Fixation Device

Feasibility of bioactive agent incorporation during device fabrication and quantity released is evidenced through the incorporation of the model antibiotic drug gentamicin and a growth factor BMP2. Gentamicin/BMP2 is encapsulated into the individual microspheres during microsphere fabrication. Gentamicin/BMP2 release pattern by interference screws in phosphate buffer solution at pH 7.4 and 37° C. is followed for 1 month.

### Evaluation of Proliferation, Differentiation and Deposition of a Mineralized Matrix of Human Mesenchymal Stem Cells (HMSC) Cultured on Bone Fixation Device

Polysaccharide and composite interference screws in vitro performance is evaluated by culturing HMSCs for up to 28 days in the presence of mineralization media to elucidate osteo-compatibility and the benefits of polysaccharide fixation device. Cellular constructs are analyzed for adhesion, proliferation and mineralization at time intervals of 1, 3, 7, 14, 21 and 28 days post-seeding.

Cell Culture:

Polysaccharide and composite bone fixation devices are incubated with 2 ml of DMEM supplemented with 10% FBS and 1% P/S in a 24 well plate at 37° C. in a humidified atmosphere. Fifty thousand HMSCs on each bone fixation device are cultured at 37° C./5% CO₂ in presence of mineralization media. Mineralization media consists of DMEM supplemented with 10% FBS, 1% P/S, 50 μg/mL ascorbate and 10 mM β-glycerophosphate. The culture media is changed twice a week. Cell adhesion and viability on the scaffolds is assessed using microscopic techniques.

Cell Proliferation:

Cell proliferation on the composite surface is determined at 1, 3, 7, 14, 21 and 28 days post-seeding by quantification of the DNA concentration (n=4 each group).

Alkaline Phosphatase Activity:

The phenotypic bone marker, alkaline phosphatase, is determined at 1, 3, 7, 14, 21 and 28 days post seeding using an alkaline phosphatase substrate kit (Bio-Rad, CA). The cell lysate obtained from the DNA assay is used to evaluate alkaline phosphatase activity (Sethuraman et al. 2007).

Alizarin Red Calcium Quantification:

Mineralized matrix synthesis by cells will be analyzed with Alizarin Red staining for calcium deposition.

### Evaluation of In Vivo Performance of Bone Fixation Devices Following Rabbit Tibia Implantation

Polysaccharide and composite interference screws are implanted in the rabbit tibia to evaluate biocompatibility, rate of new bone formation and screw integration with the surrounding tissue. Every 4 weeks, the rabbit tibia is x-rayed to determine the extent of healing. At 4 and 12 weeks, animals are sacrificed and the extent of mineralized tissue formation is quantified using micro-CT. Histological evaluation is performed by staining with Sanderson's rapid bone stain, and the mechanical properties of the defect site will be evaluated using compression testing. Two cellulose acetate (low and high molecular weight), two cellulose acetate composites (low and high molecular weight), one ethyl cellulose and one ethyl cellulose composite interference screws are evaluated in the rabbit model. Commercially available PLLA and PLLA/composite interference screw are used as controls in this model.

Rabbit Tibia Defect Model:

In this study adult New Zealand White rabbits (4-5 kg) are randomly divided into 8 groups: (1) Interference screws of CA low molecular weight (n=8), (2) Interference screws of CA high molecular weight (n=8), (3) Interference screws of EC (n=8), (4) Interference screw of commercial PLLA product (control) (n=8), (5) composite interference screws of CA selected (n=8), (6) composite interference screws of EC selected (n=8), (7) composite interference screws of commercial PLLA product (control) (n=8), and (8) defect alone (n=8) negative control. The animal study is performed in accordance with the Institutional Animal Care and Use committee regulations. Bilateral holes (6 mm hole diameter and 1.2 mm length) starting 3 mm below the joint line in the anteromedial cortex of the proximal tibia are created using a micro-burr with a 3 mm tip and saline irrigation to minimize thermal damage. Each tibia receives 2 implants 1 cm apart. X-rays will be obtained immediately post-operative and every 3 weeks thereafter. Half of the animals are sacrificed at 6 weeks and samples will be collected for microCT analysis, histological analysis, and push out tests. Remaining animals are sacrificed at 12 weeks for the analysis.

MicroCT:

The microCT (model viva CT 40; Scanco Medical, Bassersdorf, Switzerland) and accompanying analysis software are used to perform all image scanning, data processing, and analysis.

Biomechanical Testing:

Tibia is slowly thawed at 4° C. overnight and equilibrated to room temperature over several hours (n=8 each group). The implanted screw is subjected for push out test using the Instron at ambient temperature and humidity at a ramp speed of 1 mm/min.

Histological Staining:

Tibia is fixed for several weeks in 10% formalin, dehydrated through a graded ethanol series, and processed using Spurr embedding medium (n=4 each group). The undecalcified samples are ground and 6 μm-thick sections cut for histochemical analysis. Sections are stained with Sanderson's rapid bone stain (Surgipath Medical Industries, Richmond, Ill.). Images are obtained using a digital camera attached to a Zeiss Axioskop 40 microscope in conjunction with PictureFrame software.

Histomorphometry:

Tibia is fixed for several weeks in 10% formalin, dehydrated through a graded ethanol series, and processed using Spurr embedding medium (n=4 each group). The undecalcified samples are ground and 6 μm-thick sections cut for dynamic/static histomorphometry (Calcein and Xylenol Orange labels) analysis using a digital camera attached to a Zeiss Axioskop 40 microscope in conjunction with Osteomeasure software.

### EXAMPLES

### Example 1. Microsphere Fabrication

Cellulose acetate (CA) or ethyl cellulose microspheres (EC) are fabricated using an oil-in-water emulsion/solvent evaporation method. In brief, either CA or EC is dissolved in a binary solvent composition of methylene chloride:acetone (9:1) at 20% (w/v). The resulting polymer solution is slowly poured into a 1% (w/v) polyvinyl alcohol aqueous solution stirring at 250 rpm. The solvent is allowed to evaporate overnight at room temperature under constant stirring. The microspheres are collected by vacuum filtration and washed with distilled water. Microspheres are sieved and separated into different sizes based on their diameter for scaffold fabrication. Three different diameters namely >1180, 1180-850, and 850-600 μm were chosen for use in bone grafts. While particles in the diameter range of 50-100 100-150, 200-250, 250-400 μm were chosen for fabricating fixation devices.

### Example 2. Scaffold Fabrication Using Solvent/Non-Solvent Sintering

It is necessary to identify a proper solvent/non-solvent composition for each polymer at which only the microsphere surface turns rubbery to facilitate bonding with the adjacent microspheres. After several trials a solvent/non-solvent composition of 3:1 ratio of acetone:water was found to be suitable for ethylcellulose (EC) microsphere sintering while 8:2 ratio of acetonitrile:water for cellulose acetate (CA). The sieved microspheres were mixed with sintering solvent and the mixture was vortexed for five seconds. The resulting slurry was placed in a cylindrical Teflon mold with a 5 mm diameter and 10 mm height. The solvent/non-solvent mixture was allowed to evaporate in a fume hood for 30 minutes followed by vacuum-drying for an additional 24 hours. Scaffolds of 8 mm diameter and 2 mm thickness were also fabricated for in vitro cell studies. In contrast control poly(lactide-co-glycolide) (PLAGA) sintered microsphere matrices were fabricated by heating 850-600 μm in diameter microspheres at 90° C. in a stainless steel mold for 2 h. Thus formed scaffolds were named as EC-600, EC-850, EC-1180 and PLAGA (control) based on the microsphere diameter. In general, to create the microsphere slurry a minimum amount of solvent-non-solvent composition was used just to wet the microsphere surfaces.

For fabricating solid bone fixation devices such as interference screw, Teflon mold will be filled with particles in the diameter range of 50-400 μm and a volume of solvent/non-solvent (150-250 μL) in excess was added to each screw to completely melt particles into a solid structure such as interference screw.

Alternatively Teflon molds are filled with selected microspheres and 1004 of solvent/non-solvent composition is added to each scaffold. 100 μL of solvent/non-solvent composition is just sufficient enough to wet the microspheres in a mold of 5 mm diameter and 10 mm height.

### Example 3. 3-D Sintered Microsphere Characterization Morphology

3-D Composite microsphere scaffold morphology is characterized by SEM. Cylindrical scaffolds (n=3) are coated with gold using a Hummer V sputtering system (Technics, Baltimore, Md.) for 5 min. Samples are visualized on a JSM 6400 (JOEL, Boston, Mass.) at 15-20 keV and a working distance of 39-48 cm.

Scanning electron microscopy (SEM) is used to characterize the morphology of the individual microspheres and the corresponding scaffolds.

TABLE I provides a summary of the mechanical properties of cylindrical and interference screw structures fabricated from CA and CA-HA under compression, bending and torsional modes.

| | Molecular | Compressive | Compressive |
|---|---|---|---|
| Cellulose Structure | weight (Mn) | Modulus (MPa) | Strength (MPa) |
| Cylinder | 30,000 | 257 ± 22 | 16 ± 4 |
| Cylinder | 50,000 | 366 ± 21 | 33 ± 8 |
| Screw | 30,000 | 415 ± 13 | 17 ± 3 |
| Screw | 50,000 | 422 ± 17 | 28 ± 10 |
| CA-15 wt % HA Screw | 30,000 | 389 ± 16 | 14 ± 3 |
| CA-15 wt % HA Screw | 50,000 | 395 ± 20 | 17 ± 5 |
| PLGA Cylinder¹ | 90,000 | 155 ± 30 | 4 ± 1 |
| PLGA Cylinder² | 234,000 | 297 ± 22 | Not reported |
| (¹Borden et al. 2002; | | | |
| ²Kofron et al. 2007) | | | |

Samples used for mechanical testing had a length to diameter ratio 2:1. Compressive mechanical properties listed in the table below are based on a sample size of n=12. Cylindrical structures were designed to replicate the earlier reported PLGA (85:15) cylindrical structures with identical pore properties such as 33-37% porosity and 120-150 μm pore diameters to compare our results. Increase in molecular weight significantly improved compressive mechanical properties of both CA porous cylinders and screws. Further CA and their composite structures showed significantly higher compressive properties than PLGA structures. Among the various CA to HA compositions (2.5-40 wt %), 15 wt % HA composition with CA produced screws of higher compressive properties due to a homogenous distribution of nano-sized HA particles. At other compositions mechanical properties were weaker. All these values are in the mid range of human trabecular bone. CA interference screw structures tested for torsional properties at a speed of 1°/s measured an ultimate torsional load of 0.46±0.1N*m, ultimate rotation of 0.19±0.05 rad, stiffness of 0.12±0.05 Nm/degree and torsional rigidity of 585.57±272.7 N*mm2. These screws showed a flexural modulus of 2080.8±630.6 Pa in a three point bending test performed at a crosshead speed of 0.1 mm/sec. CA screw structures reached a maximum flexural stress of 142.38±10.08 N/m2 and strain of 0.077±0.004. These values were found to be better than PLA and their bioceramic screw structures reported in the literature and suited for functional graft fixation.

### Example 4. Design and Fabrication of Cellulose Based Interference Screws

A Teflon mold was custom designed to produce the screws with the dimensions of 7 mm diameter and 20 mm length to replicate the dimensions of the Smith and Nephew PLA/hydroxylapatite BIORCI and BIORCI•IA bioabsorbable screws for direct comparison purposes. Further our interference screw design included an Allen wrench style hole for driving mechanism-ensuring similarity to existing commercial products. Cellulose acetate particles in the diameter range of 100-150 μm were produced from solutions of CA using a water-in oil emulsion/solvent evaporation technique. Teflon molds were tightly filled with CA particles and a solvent composition of 3:1 ratio of Acetone:Cyclohexane was added to the mold to melt the particles to produce the screw structures at the room temperature. Screws were taken out of the mold after 3 hours and kept desiccated until further use. Preliminary results suggest intact structure and bioactivity of the encapsulated model proteins and antibiotic drugs following the screw fabrication at the room temperature. This approach is attractive for delivering growth factors and antibiotics to achieve better graft fixation.

### Example 5. Performance of Mesenchymal Stem Cells in Polysaccharide Based Screws

Inventors evaluate hMSCs performance in terms of adhesion, proliferation and differentiation on 2 mm thick discs of CA and CA-HA/TCP cut from the screws in culture. PLA and PLA-HA discs will be used as controls. Each disc is seeded with 50,000 hMSCs and cultured in a standard basal media supplemented with 10% FBS and 1% P/S. After a day one set of disc culture media will be changed to osteogenic media containing 10⁻⁸M dexamethasone, 150 μg/mL L-ascorbic acid, and 10 mM β-glycerophosphate. Cell adhesion and viability on the scaffolds will be performed using microscopic techniques. Cell proliferation is determined by quantification of the DNA concentration (n=4). The phenotypic bone marker, ALP will be determined using an ALP substrate kit (Bio-Rad). Mineralized matrix synthesis by cells will be analyzed with Alizarin Red staining for calcium deposition. Using RT-PCR, expression of type I collagen (TIC), ALP, osteocalcin (OCN), and osteopontin (OPN) by hMSCs cultured on discs is evaluated (n=4).

### Example 6. Rabbit Tibia Defect Model to Assess Osteointegration

Adult New Zealand White Rabbits (4-5 kg) are used in accordance with the Institutional Animal Care and Use Committee regulations. Due to the anatomical restrictions screw dimensions are reduced to half based on the regulatory guidelines of ASTM F981-04 and ASTM F1983-99 (2008). Further to establish the interfacial bonding between the CA structures and bone (osseointegration) cylindrical structures that avoid the influence of screw threads in determining osseointegration are used. We implant cylindrical structures of 3 mm diameter and 6 mm length in the rabbit tibia. The experimental groups include (1) CA (n=9), (2) CA-HA/TCP (n=9), (3) PLA (control) (n=9), (4) PLA-HA (control) (n=9) and (5) Defect alone (control) (n=9).

Surgical Procedure:

Bilateral holes (3 mm hole diameter and 6 mm length) starting 3 mm below the joint line in the anteromedial cortex of the proximal tibia are created using a micro-burr with a 3 mm tip and saline irrigation to minimize thermal damage. Each tibia receives 2 implants 1 cm apart. X-rays are obtained immediately post-operative and every 3 weeks thereafter. Half of the animals are sacrificed at 12 weeks and samples are collected for microCT analysis, histological analysis, and push out tests. Remaining animals are sacrificed at 24 weeks for the analysis.

Specimen Characterization:

The microCT is used to perform all image scanning, data processing, and analysis. Tibia (n=6) from each group are tested with a custom designed holder in compression using the Instron 5544 at ambient temperature and humidity at a ramp speed of 1 mm/min. Polymeric samples recovered from push out tests are subjected for changes in molecular weight and morphology. Tibia is fixed for several weeks in 10% formalin, dehydrated through a graded ethanol series, and processed using Spurr embedding medium (n=3 each group). The undecalcified samples are ground and 6μm-thick sections cut for histochemical analysis. Sections are stained with Sanderson's rapid bone stain and imaged using a Zeiss Axioskop 40 microscope in conjunction with PictureFrame software.

What is claimed is:

1. A bone fixation device comprising: a cellulose acetate-hydroxyl apatite composite, wherein the composite comprises 15% hydroxyl apatite and wherein the cellulose acetate has a molecular weight of 30,000 Da or 50,000 Da, wherein the composite is comprised of fused microspheres having a diameter of between 50 um and 400 um.

2. The bone fixation device of claim 1, wherein the composite comprises a porosity of between 33% and 37%.

3. The bone fixation device of claim 1, wherein the composite comprises pore diameters of between 120 urn and 150 um.

4. The bone fixation device of claim 1, wherein the bone fixation device is a cylindrical screw or an interference screw.

5. The bone fixation device of claim 1, wherein the cellulose acetate has a molecular weight of 50,000 Da.

6. A method of making a bone fixation device comprising providing a plurality of polysaccharide microspheres or polysaccharide particles in a mold, wherein the mold is in the form of the bone fixation device; providing a solvent system having an organic solvent fraction and an aqueous fraction dropwise in the mold; removing excess solvent from the mold; and drying the microspheres or particles in the mold.

7. A method of treating a patient in need of bone repair, comprising implanting a bone fixation device in the patient at a site of bone damage, ligament damage, or bone deformity, wherein the device consists of a cellulose acetate-hydroxyl apatite composite, wherein the composite comprises 15% hydroxyl apatite and wherein the cellulose acetate has a molecular weight of at least 30,000 Da.
